(19) United States
(12) Patent Application Publication
(10) Pub. No.: US 2012/0095487 A1
(43) Pub. Date: Apr. 19, 2012

(54) Pricking Aid Comprising A Lancet Ejection Means

(75) Inventors: Andreas Knie, Regensburg (DE); Marion Butz, Regensburg (DE); Bernhard Schmitt, Schwandorf (DE)

(73) Assignee: GERRESHEIMER REGENSBURG GMBH, Regensburg (DE)

(21) Appl. No.: 13/262,565

(22) PCT Filed: Mar. 17, 2010
(86) PCT No.: PCT/EP10/53423

§ 371 Date: Dec. 13, 2011

(30) Foreign Application Priority Data
Apr. 3, 2009 (DE) 10 2009 016 126.0
Jun. 18, 2009 (DE) 10 2009 025 444.7

(51) Int. Cl. A61B 17/32

(52) U.S. Cl. 606/182

(57) ABSTRACT

The invention relates to a blood lancet apparatus with a base member for the arrangement of a plurality of apparatus components. The base member comprises for example a lancet-receiving device for coupling the blood lancet apparatus to a lancet element, at least one spring element for acting upon the lancet-receiving device with spring force and an actuating element for initiating a movement of the lancet-receiving device. The coupling between the lancet-receiving device and the lancet element is capable of being altered by means of a fixing and/or dispensing device. According to the invention the lancet-receiving device is capable of being deformed by means of a movement of the fixing and/or dispensing device in the longitudinal direction of the lancet-receiving device and by means of an engagement of the fixing and/or dispensing device in at least one recess in the lancet-receiving device.

### CROSS-REFERENCE TO RELATED APPLICATIONS

This application is a U.S. National Stage Application under 35 U.S.C. §371 of International Application No. PCT/EP2010/053423, filed Mar. 17, 2010, which claims the benefit of German Application No. 10 2009 016 126.0, filed Apr. 3, 2009, and German Application No. 10 2009 025 444.7, filed Jun. 18, 2009. All of these applications are hereby incorporated by reference in their entireties.

### DESCRIPTION

The invention relates to a lancing device with a lancet ejection means, in particular of a fixing and/or dispensing device according to the preamble of claim 1.

Until now it has been customary for a lancet or a lancet element, which is arranged inside a lancet-receiving device inside a blood lancet apparatus for pricking the skin for the removal of blood, to be withdrawn by hand out of the casing or the lancet-holding device of the blood lancet apparatus when the lancet element is changed. A problem in this case is that there is a risk of an unintentional injury on the one hand and that it is necessary for lancet elements contaminated with blood from a piercing carried out previously to be handled on the other hand.

Accordingly, a fixing and/or dispensing device for blood lancet apparatus is to be made available, which permits an ejection of the lancet element from a lancet-receiving device without touching the actual lancet element.

The object set above is attained by the subject matter of apparatus claim 1 which describes a blood lancet apparatus. The blood lancet apparatus has a base member for the arrangement of a plurality of apparatus components. The aforesaid apparatus components comprise a lancet-receiving device, a spring element, an actuating element and a fixing and/or dispensing device. In this case the lancet-receiving device is used for coupling the lancet apparatus to a lancet element. The coupling is preferably solely with friction locking, but a connection with positive locking or a combined connection with positive locking and friction locking or non-positive locking respectively is also possible.

The at least one spring element is used for acting upon the lancet-receiving device with spring force, so that energy may be made available for the movement of the lancet-receiving device. The lancet-receiving device is coupled to a control cam in such a way that the lancet-receiving device is movable by a movement of the control cam, the control cam being coupled to the at least one spring element. As a result of initiating the movement by means of the actuating element the lancet-receiving device is capable of being moved in such a way that the lancet element moves automatically out of the apparatus in a dispensing direction or preferably out of the apparatus and in a receiving direction into the apparatus again.

The dispensing direction and the receiving direction are both orientated in the direction of a longitudinal axis of the lancet-receiving device, but the dispensing direction leads out of the blood lancet apparatus and the receiving direction leads into the blood lancet apparatus in a direction opposed to the dispensing direction.

In addition, an actuating element is provided for initiating a movement of the lancet-receiving device, the actuating element preferably being designed in the form of a push button which is capable of being actuated manually. When actuated, this push button co-operates with the control cam, as a result of which the control cam is released from a locked position in order to carry out a pivoting movement. In this case the lancet-receiving device slides on the control cam and is set in motion itself.

The coupling between the lancet-receiving device and the lancet element is capable of being altered by means of the fixing and/or dispensing device, i.e. it can preferably be changed between a connection with friction locking and an uncoupled state (no connection or a loose connection).

The co-operation of the fixing and/or dispensing device with the lancet-receiving device is carried out for example by means of non-positive locking between these devices. In this case the force is capable of being transmitted for example directly from the fixing and/or dispensing device to the lancet-receiving device or vice versa.

According to the invention the lancet-receiving device is capable of being deformed by means of a movement of the fixing and/or dispensing device in the longitudinal direction of the lancet-receiving device and by means of an engagement of the fixing and/or dispensing device in at least one recess in the lancet-receiving device, in which case the deformation preferably takes place solely resiliently and a recovery preferably likewise takes place automatically. This means that it is preferable for a non-deformed state of the lancet-receiving device, which represents a first coupling state in which the lancet element is connected in a stationary manner to the lancet-receiving device, is present in a first position of a fixing and/or dispensing device and for a deformed state, which represents a second coupling state in which the lancet element is movable relative to the lancet-receiving device, to be present in the second position.

It is likewise possible for the lancet element to be movable in the second coupling state not in the longitudinal direction but in a direction which is orientated substantially at a right angle to the longitudinal direction. In addition, a combined movement of the lancet element in the longitudinal direction and at a right angle to the longitudinal direction is possible.

The subject matter of claim 1 is advantageous with respect to the subject matters known from the prior art, since the ejection of a lancet element can take place without the user or the patient having to handle the used lancet element.

In a further preferred embodiment of the present invention a portion of the fixing and/or dispensing device is made wedge-shaped. This means that the cross-section of the wedge-shaped portion changes at least in one direction. By means of a co-operation of the wedge-shaped portion with a portion of the lancet-receiving device, a change in a cross-section of the lancet-receiving device is capable of being carried out.

The shape of the portion of the lancet-receiving device is capable of being selected as desired or of being adapted accordingly to the shape of the wedge-shaped portion. This embodiment is advantageous, since the fixing and/or dispensing device is capable of being brought by the wedge-shaped portion into contact with the portion of the lancet-receiving device in a precise manner and the force required for deforming the lancet-receiving device is capable of being transmitted in the desired direction.

In a further preferred embodiment of the present invention the lancet-receiving device is constructed at least sectionally in the form of a tube. This means that a hollow body of any desired and/or variable cross-sectional shape such as for example angular (triangular, rectangular, polygonal) or curved or preferably round is provided.

The portion of the lancet-receiving device is formed in the region of the at least one recess, preferably in the at least one recess, as a result of which the wedge-shaped portion is movable in the lancet-holding device and/or in the lancet-holding device. In addition, it is preferable for at least two recesses to be provided in the lancet-receiving device. This has the advantage that the lancet-holding device is capable of being deformed in a simpler manner. In the case of two recesses they are preferably arranged substantially opposite each other and at least one recess extends at least sectionally in the longitudinal direction and is designed in the manner of a slot. This likewise has the advantage that the discontinuation of the connection for example of the non-positive locking is carried out for example by reducing the frictional force between the lancet element and the lancet-receiving device.

In a further preferred embodiment of the present invention the wedge-shaped portion of the fixing and/or dispensing device is capable of being introduced into an indentation of the recess of the lancet-receiving device. This means that the lancet-receiving device has an indentation which allows the wedge-shaped portion to be capable of being introduced at least in part or preferably completely into the recess. In this case the slot represents a further extension of the indentation. This means that the slot and the indentation are joined to each other. This embodiment is advantageous since the wedge-shaped portion is capable of being introduced in a defined manner through the recess into the slot, and this results in a high degree of functional reliability.

In a further preferred embodiment of the present invention the wedge-shaped portion of the fixing and/or dispensing device is capable of being brought into contact at least for a time, i.e. in the coupling state, and sectionally, i.e. at least one region of the wedge-shaped portion, with the portion of the lancet-receiving device, in which case the edge of the wedge preferably extends substantially at a right angle to the longitudinal direction of the receiving element. Furthermore, a force is capable of being transmitted manually through the fixing and/or dispensing device to the lancet element. This means that the lancet element is capable of being moved by a force exerted by the user or the patient, it being possible at the same time, as a result of the application of force, for the lancet element to be moved at least in part in the dispensing direction and for the slot to be altered. An alteration of the slot corresponds to a widening and/or a reduction of the cross-section of the lancet-receiving device.

In a further preferred embodiment of the present invention the wedge-shaped portion has a pointed front region, “pointed” in this case describing a front region which has a smaller area or cross-sectional area as compared with a cross-section of a wider region of the wedge-shaped portion. In this way, the pointed front region can be made obtuse or sharp-edged. Furthermore, the front region is capable of being introduced at least sectionally into the slot, it being preferable for the height of the front region to be less than the height of the slot. In addition, the front region of the wedge-shaped portion is capable of being introduced at least sectionally into an inner space of the lancet-receiving device which receives the lancet element, as a result of which the fixing and/or dispensing device is capable of being brought into contact with the lancet element. This embodiment is advantageous since, by the contact between the wedge-shaped portion extending at least sectionally into the inner space of the lancet-receiving device and the lancet element, it likewise makes possible a transmission of force by the operator by way of the fixing and/or dispensing device to the lancet element, and this in turn makes possible a simultaneous displacement of the lancet element and a deformation of the lancet-receiving device.

In a further preferred embodiment of the present invention the fixing and/or dispensing device has a rod-shaped element, on the end of which the wedge-shaped portion is arranged, preferably offset laterally. This means that a centre of the wedge-shaped portion is preferably arranged offset with respect to a centre of the rod-shaped element. This embodiment is advantageous since a separation or a splitting of the functions of the wedge-shaped portion takes place in such a way that the portion of the wedge-shaped portion arranged directly on the rod-shaped element is capable of being used for absorbing relatively high forces and thus for deforming the lancet-receiving device, whilst the portion of the wedge-shaped portion facing away from the rod-shaped element is provided merely for ejecting or moving the lancet element. This leads to a load-compliant and an optimized design of the fixing and/or dispensing device, as a result of which the function reliability is increased and savings in material can be made.

In a further preferred embodiment of the present invention the wedge-shaped element has an underside which is arranged in a common plane with an underside of the rod-shaped element. This embodiment is advantageous since the wedge-shaped portion and the rod-shaped element can be supported by means of a wall or support in such a way that high forces and moments can act upon the rod-shaped element and the wedge-shaped portion respectively, without the loading leading to damage to the fixing and/or dispensing device.

In a further preferred embodiment of the present invention the wedge-shaped portion has an end region in addition to the pointed front region, the cross-sectional height of the pointed front region preferably being lower than the cross-sectional height of the rod-shaped element. Furthermore, the cross-sectional height of the end region is preferably higher than the cross-sectional height of the rod-shaped element, in which case it is also possible for the cross-sectional height of the end region to correspond to the cross-sectional height of the rod-shaped element or to be made lower than the cross-sectional height of the rod-shaped element. The preferred embodiment is advantageous since it leads to a reduction in the proportion of material and thus in the costs and the weight.

In a further preferred embodiment of the present invention the movement of the fixing and/or dispensing device is coupled to the casing by at least one further spring element. As a result, the coupling of the spring element to the fixing and/or dispensing device is capable of being altered in a manner dependent upon the deflection of the spring element. The spring element is guided by means provided on the fixing and/or dispensing device and/or on the base member and the fixing and/or dispensing device is capable of being moved in a direction opposed to the dispensing direction by means of the spring element. This means that during a displacement the fixing and/or dispensing device undergoes a change in the coupling to the casing or to the base member respectively.

After a release of the fixing and/or dispensing device by the user or the patient it is preferable for the fixing and/or dispensing device to be set back, as a result of which the lancet-receiving device is moved from a deformed state into a non-deformed state. This embodiment is advantageous since the slide is preferably transferred automatically by means of the spring element into a position at a distance from the lancet-receiving device and so whenever the user or the patient releases the lancet apparatus a non-deformed state of the lancet-receiving device is always made possible or is present.

In a further preferred embodiment of the present invention the fixing and/or dispensing device has provided on it a stop by means of which the displacement path of the fixing and/or dispensing device is limited and which co-operates with preferably one suitably designed or corresponding region of the base member. This has the advantage that a maximum movement of the fixing and/or dispensing device is capable of being pre-set in a defined manner. This in turn has the advantage that excessive loads of components of the blood lancet apparatus are eliminated or limited.

In a further preferred embodiment of the present invention a manual actuation element is provided on one side of the stop of the fixing and/or dispensing device. The fixing and/or dispensing device can be moved by the user or the patient respectively by means of this manual actuation element.

In a further preferred embodiment of the present invention the fixing and/or dispensing device has a carrier element which extends substantially at a right angle to the longitudinal direction and on which the means for guiding the further spring element, the bar-shaped or rod-shaped element and the stop are provided substantially at a right angle.

It is preferable for the means for guiding the further spring element, the rod-shaped element and the stop to be arranged parallel to one another.

Furthermore, reinforcement elements are provided in a preferred manner between the stop and the means for guiding the further spring element and/or between the rod-shaped element and the means for guiding the further spring element. This embodiment is advantageous since on account of the reinforcement elements comparatively high forces can be transmitted to the elements named above, without the latter having to be made extremely solid, i.e. savings in material are provided by means of optimized applications of force.

In a further preferred embodiment of the present invention at least the wedge-shaped portion is produced in one piece with the remainder of the fixing and/or dispensing device, in particular with the rod-shaped element. This is advantageous, since on account of this design the flow of force inside the fixing and/or dispensing device can pass in an optimum manner since no stressing on account of inaccuracies in terms of joins occurs.

In a further preferred embodiment of the present invention the base member has at least one flat inner wall for displacing the fixing and/or dispensing device in a defined manner. This embodiment is advantageous since the fixing and/or dispensing device can be displaced on the inner wall or a web respectively or a portion of an inner wall, and the inner wall, the portion of the inner wall or the web is likewise used for absorbing excessive actuation forces, as a result of which damage to the blood lancet apparatus is prevented.

At this point it should be mentioned that all the features disclosed in the application documents advantageously further develop, either individually or in combination with one another, generic blood lancet apparatus or blood lancet apparatus known from the prior art.

Further advantages, aims and properties of the present invention are explained with reference to the following description of accompanying drawings, in which blood lancet apparatus for the investigation of blood are illustrated by way of example. Components of the blood lancet apparatus which correspond at least substantially with respect to their function in the figures can be designated with the same references in this case, it being unnecessary for these components to be numbered or explained in all the figures. In the figures

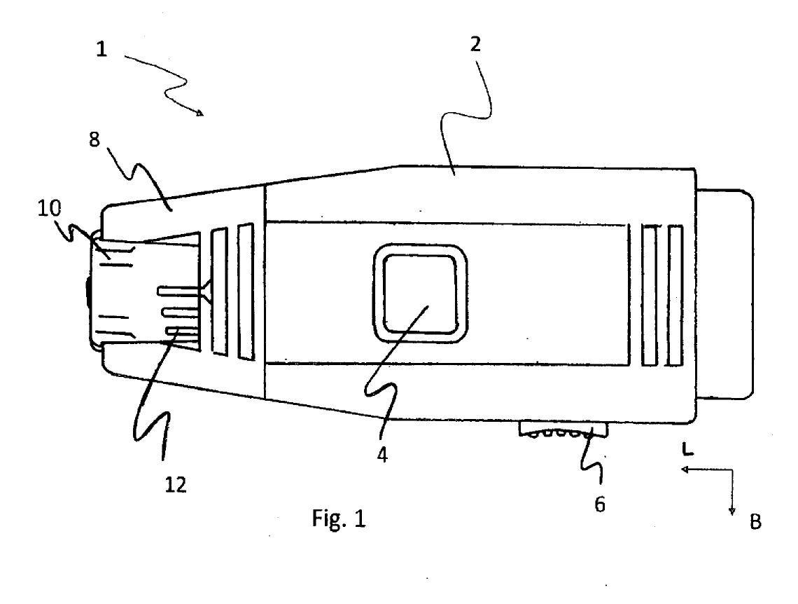

FIG. 1 is an illustration of a blood lancet apparatus according to the invention;

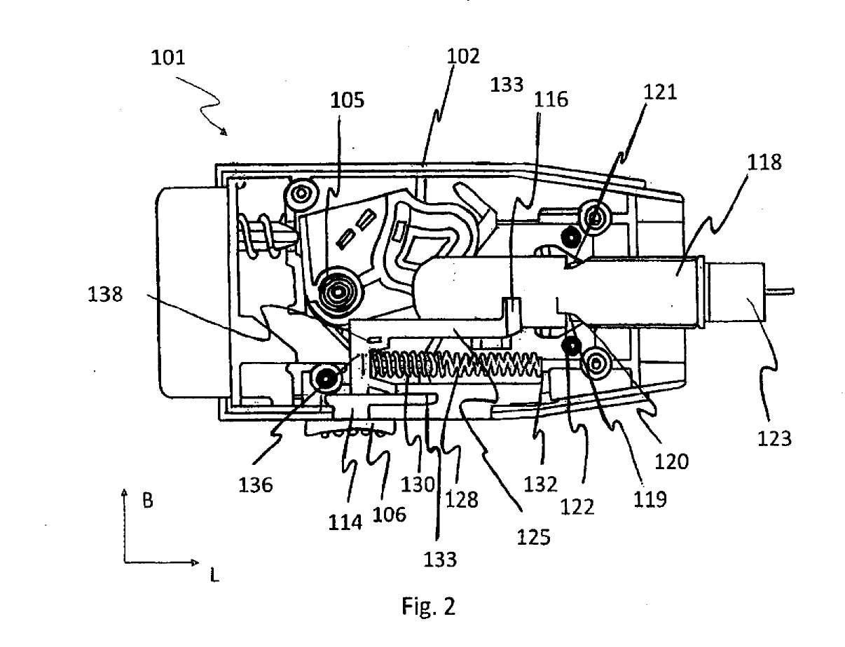

FIG. 2 is an open illustration of the blood lancet apparatus according to the invention in an uncoupled state;

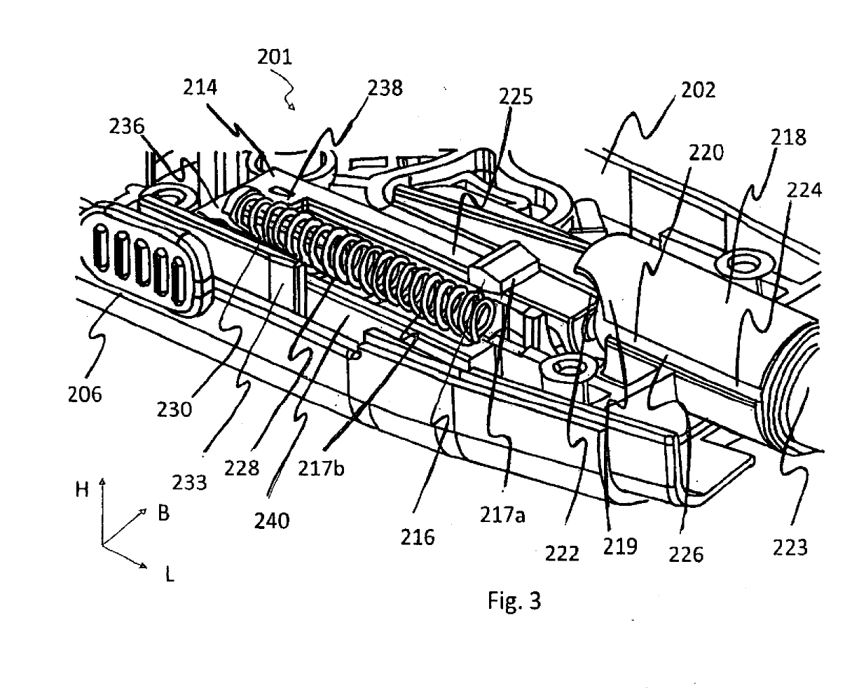

FIG. 3 is an open three-dimensional illustration of the blood lancet apparatus according to the invention in an uncoupled state;

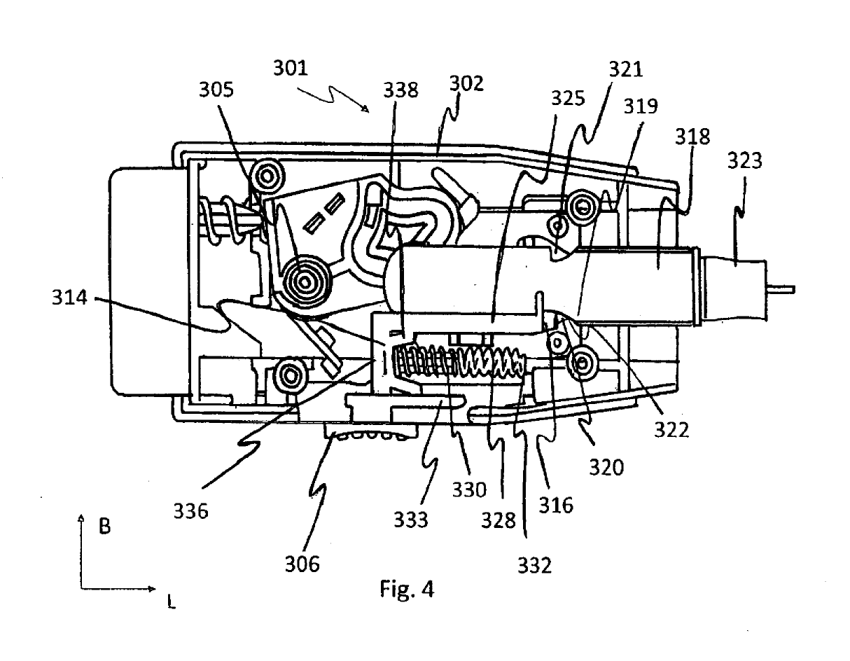

FIG. 4 is an open illustration of a blood lancet apparatus according to the invention in a coupled state;

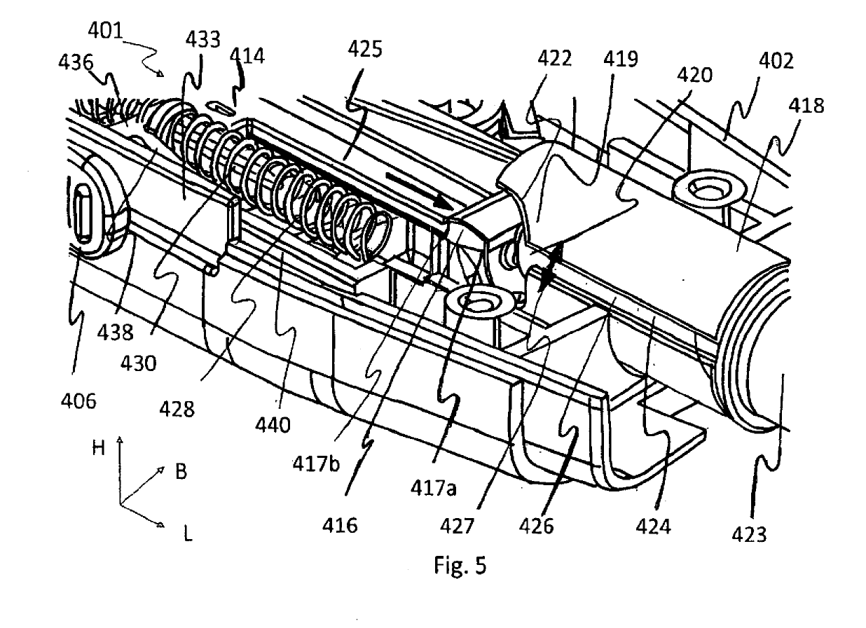

FIG. 5 is an open three-dimensional illustration of a blood lancet apparatus according to the invention in a coupled state;

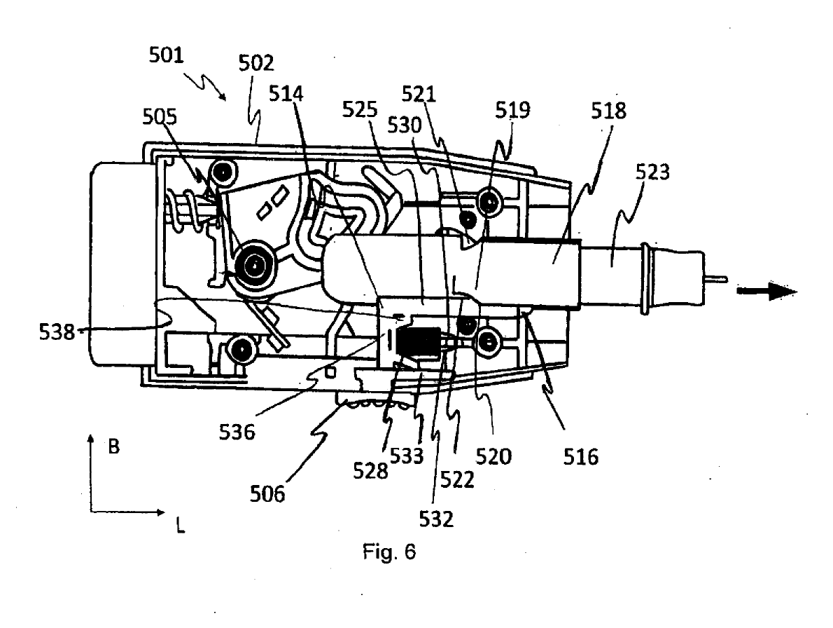

FIG. 6 is an open illustration of a blood lancet apparatus according to the invention in a dispensing configuration;

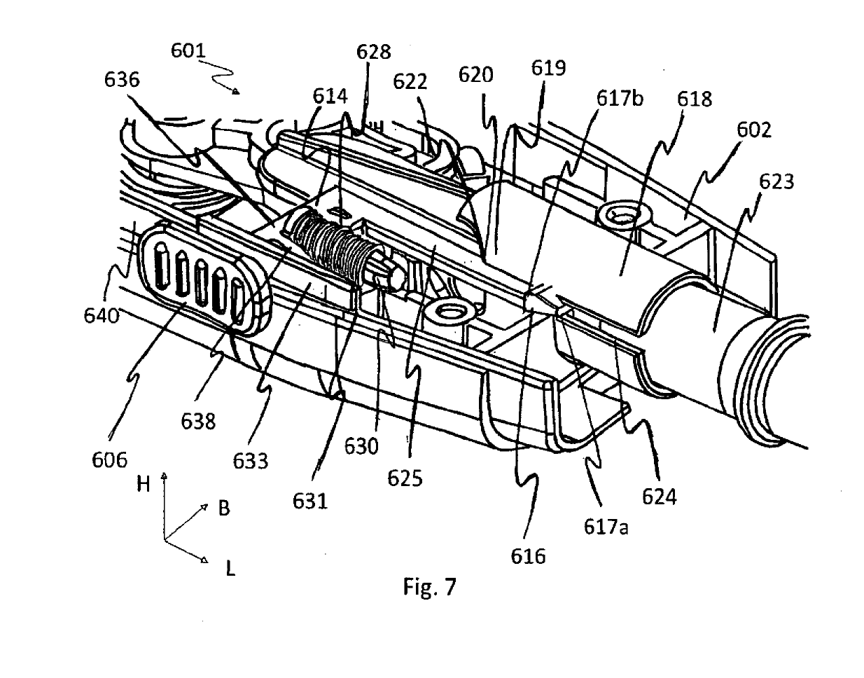

FIG. 7 is an open three-dimensional illustration of a blood lancet apparatus according to the invention in a dispensing configuration;

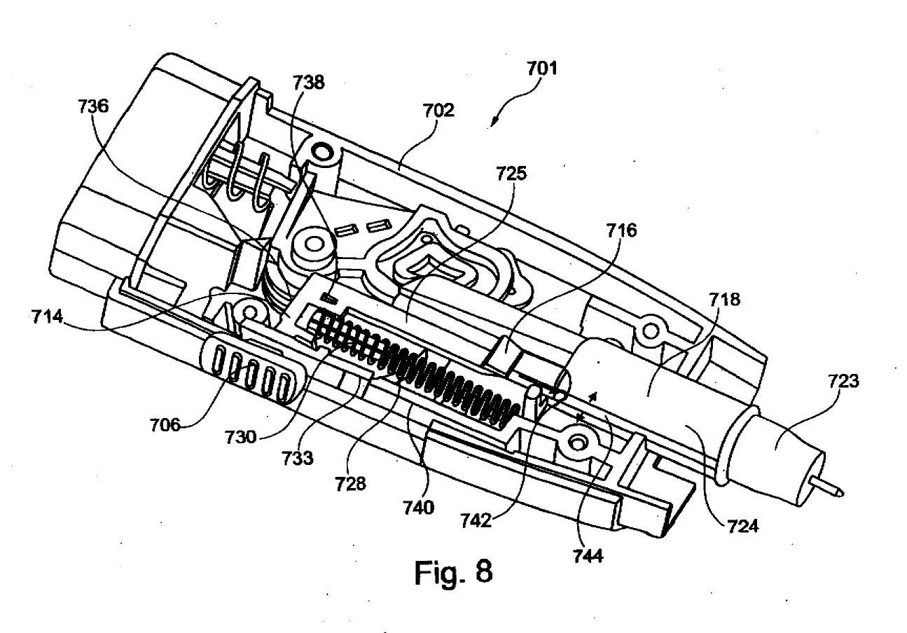

FIG. 8 is a further perspective illustration of a blood lancet apparatus according to the invention in a coupled state;

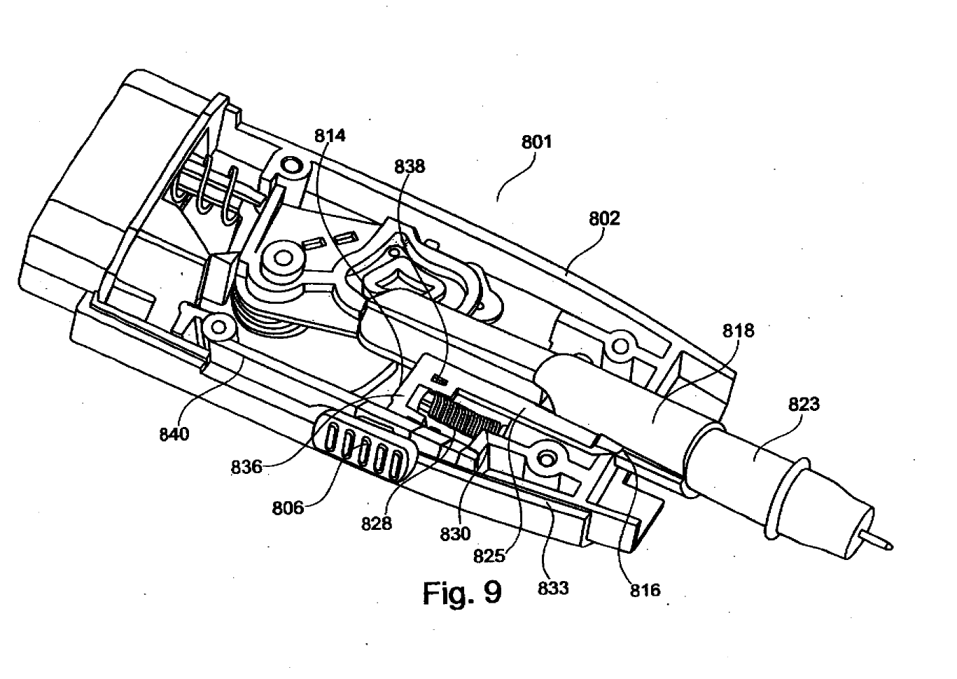

FIG. 9 is a further perspective illustration of a blood lancet apparatus according to the invention in a dispensing configuration;

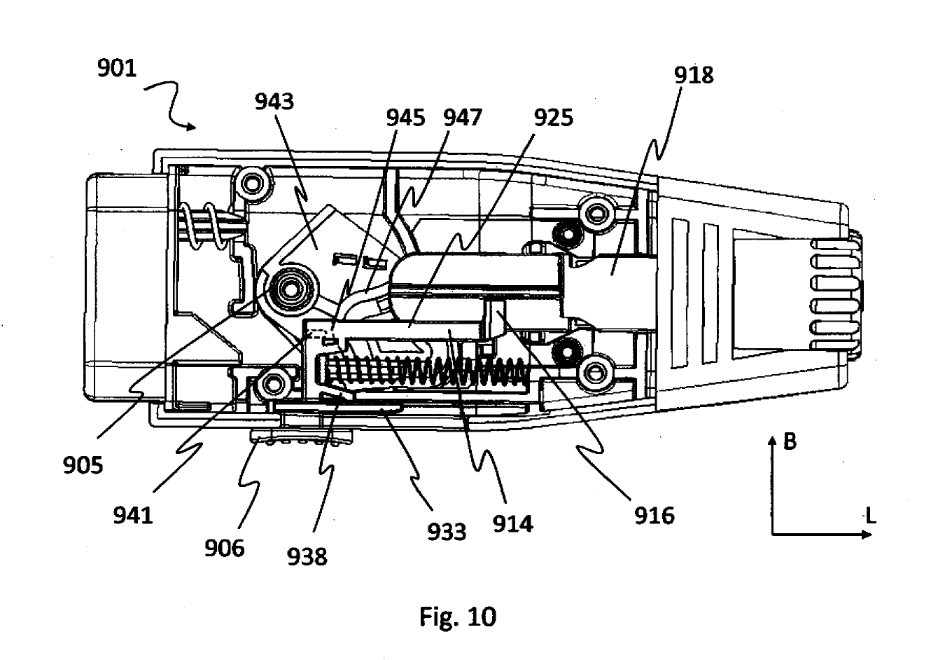

FIG. 10 is a further illustration of the blood lancet apparatus with a fixing and/or dispensing device locked, and

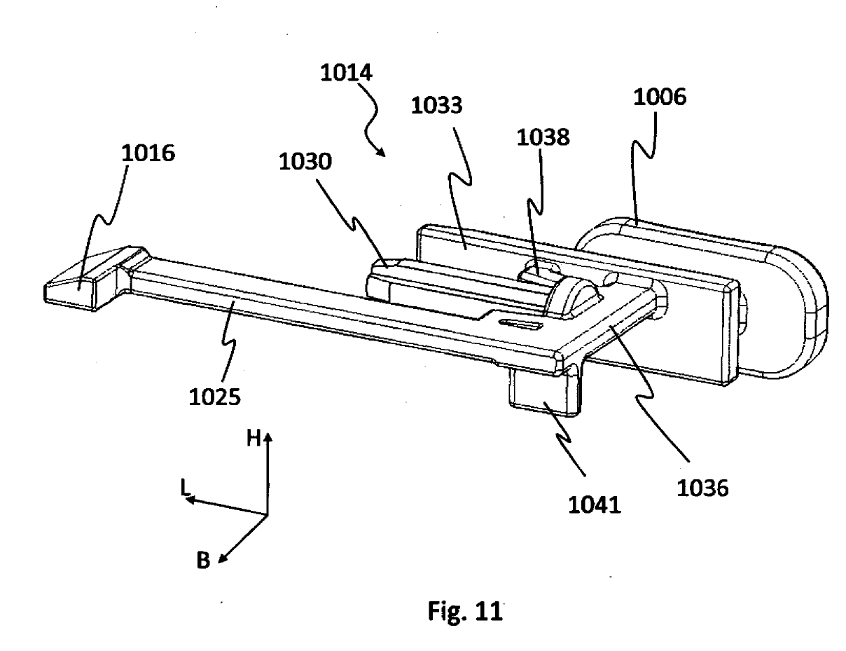

FIG. 11 is a three-dimensional illustration of the fixing and/or dispensing device.

FIG. 1 shows a blood lancet apparatus 1 with a base member 2 which has an actuation element 4 for initiating the function of the blood lancet apparatus 1 and a manual actuation element 6 for the ejection of lancet elements (not shown in this illustration) and a cap 8 on which is provided a setting wheel 10, by means of which a setting of the pricking depth is capable of being carried out. In this case the pricking depth is capable of being set in stages by means of markings 12 on the setting wheel 10 and the cap 8. The blood lancet apparatus 1 extends in the longitudinal direction L and the width-wise direction B.

In FIG. 2 a blood lancet apparatus 101 is shown which likewise extends in the longitudinal direction L and the width-wise direction B. The blood lancet apparatus 101 has a base member 102 in which a plurality of apparatus components are arranged. A force for the function execution is capable of being transmitted to the lancet-receiving device 118 by means of a spring element 105 and a control cam.

A wedge-shaped portion 116 can be displaced in the longitudinal direction L of the blood lancet apparatus 101 by means of a manual actuation element 106 which is arranged on a fixing and/or dispensing device 114 or which is joined to the latter in one piece.

A portion 119, on which or with which the wedge-shaped portion 116 can be brought into engagement or contact, is provided on the lancet-receiving device 118. In particular, the portion 119 of the lancet-receiving device 118 is provided in a recess 120 in the lancet-receiving device 118.

In addition to a first recess 120 the lancet-receiving device 118 like-wise has a second recess 121. An indentation 122 is preferably provided in at least one of the two recesses 120, 121. The wedge-shaped portion 116 is capable of being introduced at least sectionally into this indentation 122. Furthermore, the wedge-shaped portion 116 is capable of being supplied to the indentation 122 in a defined manner.

A lancet element 123 is capable of being arranged in the lancet-receiving device 118, and, in addition, the fixing and/or dispensing device 114 has a rod-shaped element 125 and it is preferably surrounded at least locally by a further spring element 128 which is supported on a means of the fixing and/or dispensing device 130 or is guided by it.

This means 130 of the fixing and/or dispensing device 114 is preferably formed in one piece with the remainder of the fixing and/or dispensing device 114 and extends in a substantially cylindrical manner in the longitudinal direction of the blood lancet apparatus 101. The further spring element 128 is additionally in contact with means 132 attached to the base member 102 or is supported on them.

In addition, the fixing and/or dispensing device 114 comprises a stop 133 which is likewise designed in one piece with the remainder of the fixing and/or dispensing device 114. This stop 133 is used to ensure a limitation of the movement of the fixing and/or dispensing device 114 in the longitudinal direction L, but it is possible in this case for a limitation of the displacement movement of the fixing and/or dispensing device 114 likewise or alternatively to be carried out by way of a further device (not shown).

The means 130 of the fixing and/or dispensing device 114 as well as the rod-shaped element 125 are arranged substantially at a right angle to a carrier element 136. This carrier element 136 is likewise a component part of the fixing and/or dispensing device 114. In addition, the means 130 of the fixing and/or dispensing device 114 as well as the rod-shaped element 125 are connected to the carrier element 136 by means of reinforcement elements 138. An increase in the loading capacity of the fixing and/or dispensing device 114 is carried out at least sectionally by the reinforcement elements 138.

FIG. 3 shows in a perspective view a cross-section of a blood lancet apparatus 201 which extends in the longitudinal direction L, the width-wise direction B and the height direction H. The blood lancet apparatus 201 likewise has a base member 202 in which a plurality of apparatus components are arranged. In addition, a manual actuation element 206 is provided, on which raised portions which increase the friction and/or generate positive locking are arranged.

The fixing and/or dispensing device 214 has a wedge-shaped portion 216 on which a front region 217a and an end region 217b are formed. The front region 217a has a smaller cross-sectional area than the end region 217b.

The lancet-receiving device 218 is made tubular in a first portion and plate-shaped in a further portion, it being preferable for two plate elements to be arranged at a right angle to each other, in order to obtain an increase in the structural stability. Furthermore, the lancet-receiving device 218 is in contact with a control cam by way of a connecting element.

A portion 219 of the lancet-receiving device 218 has a recess 220 provided on it, which preferably has an indentation 222 in which the wedge-shaped portion 216 can engage and which is preferably followed by a slot which extends in the longitudinal direction L of the blood lancet apparatus 201 or the lancet-receiving device 218, respectively. A lancet element 223 is fixed with friction locking inside the lancet-receiving device 218.

The wedge-shaped portion 216 is displaceable in the longitudinal direction L with the aid of the rod-shaped element 225 on which the aforesaid wedge-shaped portion 216 is arranged. The wedge-shaped portion 216 is capable of being introduced by a longitudinal displacement thereof into the interior 226 of the lancet-receiving device 218. A longitudinal displacement of the wedge-shaped portion 216 means a longitudinal displacement of the fixing and/or dispensing device 214, as a result of which the further spring element 228 is acted upon with force. A force is thus exerted by the spring element 228 upon the fixing and/or dispensing device 214, by which a reverse adjustment of the fixing and/or dispensing device 214 is capable of being transferred into a starting position. An uncoupled state is present in this starting position and the wedge-shaped portion 216 does not cause any deformation of the lancet-receiving device 218.

The stop 233 rests with its underside preferably flat on an inner wall 240, as a result of which forces transmitted to the fixing and/or dispensing device 214 in the width-wise direction B are supported on the casing 202 or the inner wall 240 respectively by means of the manual actuation element 206. Furthermore, the inner wall 240 forms a sliding face on which the stop 233 is displaceable.

A blood lancet apparatus 301, in which the fixing and/or dispensing device 314 is displaced in part, is illustrated in FIG. 4. In the position illustrated, the wedge-shaped portion 316 of the fixing and/or dispensing device 314 enters an indentation 322 formed at the rear of the lancet-receiving device 318 and starts to push the lancet element 323 at the rear. At the same time the wedge-shaped element 316 is brought in a deliberate manner through the region 319 in the indentation 322 to the slot (not shown) and an initial spreading of the lancet-receiving device 318 takes place. In this way, during the displacement movement illustrated here the fixing and/or dispensing device 314 first moves the rear side or the bottom of the lancet element 323 and presses the latter with the front region 317a out of the blood lancet apparatus 301 or the lancet-receiving device 318 respectively in the longitudinal direction L during the displacement movements.

After that, the laterally slotted lancet-receiving device 318 is additionally or still pressed/pushed open with the aid of the sliding element or the fixing and/or dispensing device 314 and the clamping of the lancet element 323 over the periphery of the lancet-receiving device 318 is discontinued. Furthermore, the further spring element 328 is partly compressed in this illustration.

An open three-dimensional illustration of the blood lancet apparatus 401 is shown in FIG. 5. A deformation or extension movement of the blood lancet-receiving device 418 during an insertion procedure is illustrated by means of the double arrow 427. It is evident that the wedge-shaped portion 416 engages in the indentation 422 and extends at least in part into the inner space 426 of the lancet-receiving device 418. The wedge-shaped portion 416 is advanced in a defined manner through the indentation 422 into the slot 424, in which case an ejection of the lancet element 423 and a spreading of the lancet-receiving device 418 preferably take place even during this insertion movement.

A blood lancet apparatus 501 is shown in FIG. 6, in which the discontinuation of the clamping and the pressing/pushing open of the lancet-receiving device 518 with the aid of the slide element or the fixing and/or dispensing device 514 respectively is clearly illustrated. In this case the fixing and/or dispensing device 514 is in an end stop on which the stop 533 is in contact with the base member 502. An ejection of the lancet element 523 by means of the fixing and/or dispensing device 514 thus takes place. The further spring element 528, which is designed in the form of a compression spring, is completely compressed in this end stop position. The wedge-shaped portion 516 of the fixing and/or dispensing device 514 is now completely inside the slot (not shown) at least in the longitudinal direction L. The slot or the lancet-receiving device 518 respectively is widened in this position in such a way that the lancet element 523 can fall out of the lancet-receiving device 518 preferably even without any further application of force by the fixing and/or dispensing device 514, without the lancet element 523 having to be handled manually. This falling-out is preferably carried out or at least assisted by pressing/pushing the fixing and/or dispensing device 514 from the rear, the fixing and/or dispensing device 514 with the wedge-shaped portion 516 at the same time causing a widening of the lancet-receiving device 518 or the lancet holder respectively and an ejection of the lancet element 523 from the lancet-receiving device 518.

A further open three-dimensional illustration of the blood lancet apparatus 601 is shown in FIG. 7. It is evident from this figure that the fixing and/or dispensing device 614 is at least in part in contact with the lancet-receiving device 618 by means of the wedge-shaped portion 616 and the rod-shaped element 625 and that it widens them. Furthermore, it is evident that the front region 617a of the wedge-shaped portion 616, which extends into the inner space 624 of the lancet-receiving device 618, causes the lancet element 623 to be ejected.

In addition, it is evident that the further spring element 628 is in a compressed state. In this case an orientation or stabilization of the spring element 628 is provided by the means 630. The stop 633 is connected in a positively locking manner to a stop 631 which is formed on the base body 602.

A further perspective illustration is reproduced in FIG. 8, in which the fixing and/or dispensing device 714 is in a displacement movement which has just started, as indicated by the arrow 742. The double arrow 744 shows that the lancet-receiving device 718 is just beginning to widen.

In FIG. 9 the fixing and/or dispensing device 814 with the manual actuation element 806 is already in the furthest forward position and in this case it has completely widened the lancet-receiving device 818 by means of the wedge-shaped portion 816. Removal of the lancet element 823 is thus possible by the released clamping connection. To this end the fixing and/or dispensing device 814 pushes the lancet element 823 preferably up to half-way out of the lancet-receiving device 818 while at the same time the lancet-receiving device is widened, in order to permit the ejection of the lancet element 823. After that, a return movement of the fixing and/or dispensing device 814 into a starting position takes place. This is carried out by means of the compression spring or the further spring element 828.

All the features disclosed in the application documents are claimed as being essential to the invention, insofar as they are novel either individually or in combination as compared with the prior art.

FIG. 10 shows a state of the blood lancet apparatus 901 in which the spring element 905, which is coupled to the control cam element 943, is stressed, i.e. a pricking movement of the blood lancet apparatus 901 is capable of being initiated. In this case the control cam element 943 has a cam path 947 in which the lancet-receiving device 918 is guided or mounted in a sliding manner by means of a pin element (not shown). A pivoting movement of the control cam element 943 thus leads to a linear movement of the lancet-receiving device 918 in the longitudinal direction L. In the state shown in FIG. 10 a locking or a limitation of the displacing movement of the fixing and/or dispensing device 914 takes place as a result of the co-operation of the wall 945, which forms the cam path 947 at least in part, with a pin element 941 (indicated with broken lines, since it is situated on the underside of the fixing and/or dispensing device in this illustration), which is arranged on the fixing and/or dispensing device 914. The limitation of the displacing movement or the locking of the fixing and/or dispensing device 914 takes place by means of positive locking, i.e. the wall 945 is in the longitudinal direction L in the state illustrated, i.e. in the ejection direction, in front of the pin element 941 and thus it forms a stop for the pin element 941. By initiating the pricking movement a deflection of the control cam element 943 takes place on account of the energy stored in the spring element 905, as a result of which the aforesaid control cam element 943 moves out of the displacement region of the fixing and/or dispensing device 914 and, as a result, permits a displacement of the fixing and/or dispensing device 914. The blood lancet apparatus 901 is thus designed in such a way that only in a state of the spring element 905 which is non-stressed or released at least in part is a displacement of the fixing and/or dispensing device 914 possible.

FIG. 11 is a three-dimensional illustration of the fixing and/or dispensing device 1014 which is preferably produced in one piece and from plastics material. The fixing and/or dispensing device 1014 has a manual actuation element 1006, a stop 1033, a carrier element 1036, on which a means 1030 and two reinforcement elements 1038 are arranged, and a rod-shaped element 1025, on which the wedge-shaped portion 1016 is arranged. Furthermore, in the region of the connection point or the transition region between the carrier element 1036 and the rod-shaped element 1025 a pin element 1041 extends substantially in the height direction H and in the width-wise direction B. In this case it is likewise possible, however, for the pin element 1041 to be produced in a deviating form if a co-operation of the pin element 1041 with the wall 947 (cf. FIG. 10) is possible.

### LIST OF REFERENCES

| | |
|---|---|
| 1, 101, 201, 301, 401, 501, 601, 701, 801, | blood lancet apparatus |
| 901 | |
| 2, 102, 202, 302, 402, 502, 602, 702, 802 | base member |
| 4 | actuation element |
| 105, 305, 505, 905 | spring element |
| 6, 106, 206, 306, 406, 506, 606, 706, 806, | manual actuation |
| 906, 1006 | element |
| 8 | cap |
| 10 | setting wheel |
| 12 | marking |
| 114, 214, 314, 414, 514, 614, 714, 814, 914, | fixing and/or |
| 1014 | dispensing device |
| 116, 216, 316, 416, 516, 616, 716, 816, 916, | wedge-shaped portion |
| 1016 | |
| 217a, 417a, 617a | slot-like front region |
| 217b, 417b, 617b | end region |
| 118, 218, 318, 418, 518, 618, 718, 818, 918 | lancet-receiving device |
| 119, 219, 319, 419, 519, 619 | portion of the lancet- |
| | receiving device |
| 120, 220, 320, 420, 520, 620 | recess |
| 121, 321, 521 | second recess |
| 122, 222, 322, 422, 522, 622 | indentation |
| 123, 223, 323, 423, 523, 623, 723, 823 | lancet element |
| 224, 424, 624, 724 | slot |
| 125, 225, 325, 425, 525, 625, 725, 825, 925, | rod-shaped element |
| 1025 | |
| 226, 426 | inner space of the |
| | lancet-receiving device |
| 427 | double arrow |
| 128, 228, 328, 428, 528, 628, 728, 828 | further spring element |
| 130, 230, 330, 430, 530, 630, 730, 830, 1030 | means of the fixing |
| | and/or dispensing |
| | device |
| 631 | stop on the base |
| | member |
| 132, 332, 532 | means attached to the |
| | base member |
| 133, 233, 333, 433, 533, 633, 733, 833, 933, | stop |
| 1033 | |
| 136, 236, 336, 436, 536, 636, 736, 836, 1036 | carrier element |
| 138, 238, 338, 438, 538, 638, 738, 838, 938, | reinforcement elements |
| 1038 | |
| 240, 440, 640, 740, 840 | inner wall |
| 941, 1041 | pin element |
| 742 | arrow |
| 943 | control cam element |
| 744 | double arrow |
| 945 | wall |
| 947 | cam path |
| L | longitudinal direction |
| B | width-wise direction |
| H | hight direction |

We claim:

1. A blood lancet apparatus with a base member for the arrangement of a plurality of apparatus components comprising a lancet-receiving device for coupling the blood lancet apparatus to a lancet element, at least one spring element for acting upon the lancet-receiving device with spring force and an actuating element for initiating a movement of the lancet-receiving device, wherein coupling between the lancet-receiving device and the lancet element is capable of being altered by means of a fixing and/or dispensing device, wherein the lancet-receiving device is capable of being deformed by means of a movement of the fixing and/or dispensing device in the longitudinal direction of the lancet-receiving device and by means of an engagement of the fixing and/or dispensing device in at least one recess in the lancet-receiving device.

2. The blood lancet apparatus according to claim 1, wherein a portion of the fixing and/or dispensing device is made wedge-shaped, and by means of a co-operation of the wedge-shaped portion with a section of the lancet-receiving device a change in a cross-section of the lancet-receiving device is capable of being carried out.

3. The blood lancet apparatus according to claim 2, wherein the lancet-receiving device is constructed at least sectionally in the form of a tube and the portion of the lancet-receiving device is formed in the region of the at least one recess, wherein at least two recesses, which are arranged opposite each other, are provided in the lancet-receiving device, and at least one recess extends at least sectionally in the longitudinal direction and is designed in the manner of a slot.

4. The blood lancet apparatus according to claim 3, wherein the wedge-shaped portion of the fixing and/or dispensing device is capable of being introduced into an indentation of the recess of the lancet-receiving device, and the slot represents a further extension of the indentation.

5. The blood lancet apparatus according to claims 3, wherein the wedge-shaped portion of the fixing and/or dispensing device is capable of being brought into contact at least for a time and sectionally with the portion of the lancet-receiving device, and a force is capable of being transmitted manually through the fixing and/or dispensing device to the lancet element, wherein as a result of the application of the force it is possible at the same time for the lancet element to be moved at least in part in the dispensing direction and for the slot to be altered.

6. The blood lancet apparatus according to claims 3, wherein the wedge-shaped portion has a pointed front region which is capable of being introduced at least sectionally into the slot, and it is capable of being introduced at least sectionally into an inner space of the lancet-receiving device which receives the lancet element.

7. The blood lancet apparatus according to claims 2, wherein the fixing and/or dispensing device has a rod-shaped element, on the end of which the wedge-shaped portion.

8. The blood lancet apparatus according to claim 7, wherein the wedge-shaped portion has an underside which is situated in a common plane with an underside of the rod-shaped element.

9. The blood lancet apparatus according to claim 7, wherein the wedge-shaped portion has an end region in addition to the pointed front region, wherein the cross-sectional height of the pointed front region is lower than the cross-sectional height of the rod-shaped element, the cross-sectional height of the end region is higher than the cross-sectional height of the rod-shaped element.

10. The blood lancet apparatus according to claim 1, wherein the movement of the fixing and/or dispensing device is coupled to the base member, by at least one further spring element, the spring element is guided by means provided on the fixing and/or dispensing device and/or on the base member and the fixing and/or dispensing device is capable of being moved in a direction opposed to the dispensing direction by means of the spring element.

11. The blood lancet apparatus according to claim 1, wherein the fixing and/or dispensing device has provided on it a stop which co-operates with the base member in such a way that the displacement path of the fixing and/or dispensing device is limited.

12. The blood lancet apparatus according to claim 11, wherein a manual actuation element is provided on one side of the stop of the fixing and/or dispensing device.

13. The blood lancet apparatus according to claim 1, wherein the fixing and/or dispensing device has a carrier element which extends substantially at a right angle to the longitudinal direction and on which means for guiding a further spring element, a rod-shaped element a stop are provided substantially at a right angle, wherein reinforcement elements are provided between the stop and the means for guiding the further spring element and/or between the rod-shaped element and the means for guiding the further spring element.

14. The blood lancet apparatus according to claim 2, wherein at least the wedge-shaped portion is produced in one piece with the remainder of the fixing and/or dispensing device.

15. The blood lancet apparatus according to claim 1, wherein the base member has at least one flat inner wall for displacing the fixing and/or dispensing device in a defined manner.

16. The blood lancet apparatus according to claim 8, wherein the wedge-shaped portion has an end region in addition to the pointed front region, wherein the cross-sectional height of the pointed front region is lower than the cross-sectional height of the rod-shaped element, and the cross-sectional height of the end region is higher than the cross-sectional height of the rod-shaped element.

17. The blood lancet apparatus according to claim 7, wherein the wedge-shaped portion is produced in one piece with the rod-shaped element.

18. The blood lancet apparatus according to claim 2, wherein the fixing and/or dispensing device has a rod-shaped element, on the end of which the wedge-shaped portion is offset laterally.
